(19) United States
(12) Patent Application Publication
(10) Pub. No.: US 2018/0353637 A1
(43) Pub. Date: Dec. 13, 2018

(54) METHOD FOR MANUFACTURING AN ANTIMICROBIAL COMPOSITION WITH A HIGH BIOCOMPATIBILITY

(71) Applicant: Feng Chia University, Taichung City (TW)

(72) Inventor: Tse-Hao KO, Taichung (TW)

(73) Assignee: Feng Chia University, Taichung City (TW)

(21) Appl. No.: 15/616,217

(22) Filed: Jun. 7, 2017

(51) Int. Cl. A61L 15/18; A61L 15/44; A61L 15/60; A61L 15/58; A61L 15/40; A61L 15/26; A61L 15/20; A61L 15/28; A61K 33/38; A61K 47/02; A61F 13/00; A61F 13/02

(52) U.S. Cl. CPC A61L 15/18; A61L 15/44; A61L 15/60; A61L 15/58; A61L 15/40; A61L 15/26; A61L 2300/104; A61L 15/28; A61K 33/38; A61K 47/02; A61F 13/00987; A61F 13/0206; A61F 13/00063; A61L 15/20

(57) ABSTRACT

A method for manufacturing an antimicrobial composition includes: providing an aqueous solution containing an active ingredient salt and a reductant; immersing a carbonaceous material in the aqueous solution; thermally drying the aqueous solution at 80-120° C. so as to attach the active ingredient salt to the carbonaceous material; and pyrolyzing the active ingredient salt at 600-800° C. to convert into a particle of the active ingredient attached to the carbonaceous material so as to form the antimicrobial composition.

### FIELD OF THE INVENTION

The present invention is directed to a method for manufacturing an antimicrobial composition, and more particularly to a method for manufacturing an antimicrobial composition with a high biocompatibility.

### BACKGROUND OF THE INVENTION

U.S. Pat. No. 7,803,424 discloses a method for manufacturing a metal-carrying carbonaceous material, and the method comprises: impregnating a carbonaceous material in an aqueous solution containing a metal-containing compound; and thermally treating the impregnated carbonaceous material at a temperature of at least 120° C. but not higher than the melting point of the metal under vacuum or in the presence of an inert gas. The present inventor discovered that the metal-carrying carbonaceous material manufactured by the foregoing method has antibacterial activity but is harmful to a human body or an animal body. When such metal-carrying carbonaceous material is prepared as a dressing, it can retard wound healing. The present inventor also pointed out that the harm to the living body results from that the manufacturing method cannot lead to a uniform distribution of the metals on the carbonaceous material. As such, the metals are aggregated on a certain area of the carbonaceous material at an excess amount, and this can elicit biological toxicity or allergy so as to harm the living body when the metal-carrying carbonaceous material is brought into contact with the living body.

Therefore, an improvement on the foregoing manufacturing method is desirable.

### SUMMARY OF THE INVENTION

A first objective of the present invention is to provide a method for manufacturing an antimicrobial composition, and the method includes: providing an aqueous solution containing an active ingredient salt and a reductant; immersing a carbonaceous material in the aqueous solution; thermally drying the aqueous solution at 80-120° C. so as to attach the active ingredient salt to the carbonaceous material; and pyrolyzing the active ingredient salt at 600-800° C. to convert into a particle of the active ingredient attached to the carbonaceous material so as to form the antimicrobial composition.

A second objective of the present invention is to provide a dressing, and the dressing sequentially includes: an anti-adhesive layer, an antimicrobial layer containing the antimicrobial composition manufactured by the foregoing method, and an absorption layer.

A third objective of the present invention is to provide a dressing, and the dressing sequentially includes: a release layer, an anti-adhesive layer, an antimicrobial layer containing the antimicrobial composition manufactured by the foregoing method, an absorption layer, and an adhesive layer, wherein the absorption layer is in contact with a first portion of the adhesive layer, a first portion of the release layer is in contact with the anti-adhesive layer, and a second portion of the release layer is in contact with a second portion of the adhesive layer.

### BRIEF DESCRIPTION OF THE DRAWINGS

FIG. 1 is a flowchart illustrating a method for manufacturing a dressing of an embodiment according to the present invention;

FIG. 2 is a schematic diagram showing a non-adhesive dressing;

FIG. 3 is a schematic diagram showing an adhesive dressing;

FIG. 4 is a scanning electron microscopic picture showing a composition obtained in Example 1; and

FIG. 5 is a scanning electron microscopic picture showing a composition obtained in Comparative Example 1.

### DETAILED DESCRIPTION OF THE INVENTION

The detailed description and preferred embodiments of the invention will be set forth in the following content, and provided for people skilled in the art so as to understand the characteristics of the invention.

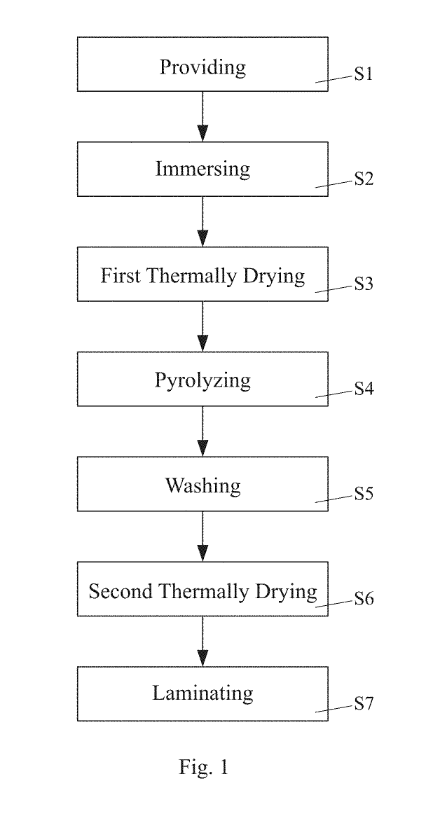

An embodiment of the present invention provides a method for manufacturing a dressing. The method can make an active particle uniformly distributed on a carbonaceous material. By such a way, the dressing can eliminate bacteria without harming a living body. As shown in FIG. 1, the method comprises the following steps of: providing (S1), immersing (S2), first thermally drying (S3), pyrolyzing (S4), washing (S5), second thermally drying (S6), and laminating (S7).

First, the providing step (S1) is providing an aqueous solution, and the aqueous solution contains an active ingredient salt and a reductant. An example of the salt is but not limited to a silver salt, a copper salt, a gold salt, a palladium salt, a zinc salt, a platinum salt, an aluminum salt, a nickel salt, a cobalt salt, a silicon salt, a calcium salt, a titanium salt, or a chromium salt. For example, the salt is a halide of the active ingredient (e.g. silver fluoride, silver chloride, silver bromide, or silver iodide), an acetate of the active ingredient (e.g. silver acetate), a nitrate of the active ingredient (e.g. silver nitrate, copper nitrate, or zinc nitrate), a phosphate of the active ingredient (e.g. silver phosphate), or a sulfonate of the active ingredient (e.g. silver sulfonate). An example of the reductant is but not limited to glacial acetic acid, ammonia water, ascorbic acid, or glucose.

Next, the immersing step (S2) is immersing a carbonaceous material in the aqueous solution, which can make the carbonaceous material contact the salt. When this step (S2) is practiced, the carbonaceous material may be soaked in the aqueous solution under a stir for 0.5-24 hours, and preferably for 1-12 hours. Furthermore, an example of the carbonaceous material is but not limited to an activated carbon fiber, a carbon fiber, an activated carbon powder, a charcoal, a bamboo charcoal granule, a carbon black, a graphite powder, a carbon nanotube, a carbon nanopowder, a graphene, a swelling graphite powder, a carbon powder made from phenol formaldehyde resins, or a carbon powder made from artificial resins. The BET specific surface area of the carbonaceous material may be of 400-2,500 m²/g, and preferably of 600-1,800 m²/g. Based on the total weight of the aqueous solution and the carbonaceous material, the salt may be present in an amount of 0.01 wt %-1 wt %, the reductant may be present in an amount of 1 wt %-50 wt %, and the carbonaceous material may be present in an amount of 0.01 wt %-4 wt %.

Afterward, the first thermally drying step (S3) is thermally drying the aqueous solution at 80-120° C., which can remove the liquid from the aqueous solution so as to attach the active ingredient salt to the carbonaceous material. When this step (S3) is practiced, the aqueous solution may be stayed at the same temperature for 0.5-6 hours, and preferably for 1-4 hours.

Then, the pyrolyzing step (S4) is pyrolyzing the active ingredient salt at 600-800° C., which can convert the salt into a particle of the active ingredient attached to the carbonaceous material so as to form an antimicrobial composition. That is, the antimicrobial composition comprises the carbonaceous material and the particle thereon. Based on the weight of the carbonaceous material, the particle may be present in an amount of 0.001 wt %-20 wt %. When the step (S4) is executed, the salt may be stayed at the same temperature for 1-6 hours. Furthermore, the step (S4) may be practiced under a vacuum, in the presence of a nitrogen gas, or in the presence of an inert gas.

Subsequently, the washing step (S5) is washing the antimicrobial composition with water, which can remove any dissociated particle from the antimicrobial composition. When this step (S5) is performed, the antimicrobial composition may be soaked in the water or rinsed with the water for 0.5-6 hours.

After which, the second thermally drying step (S6) is thermally drying the antimicrobial composition at 80-120° C., which can remove any remaining liquid from the antimicrobial composition. When this step (S6) is practiced, the antimicrobial composition may be stayed at the foregoing temperature for 0.5-6 hours, and preferably for 1-4 hours.

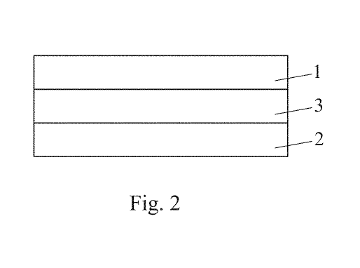

Finally, the laminating step (S7) can be depositing an antimicrobial layer (3) containing the antimicrobial composition between an absorption layer (1) and an anti-adhesive layer (2) to form a non-adhesive dressing (FIG. 2). When the dressing is applied on a wound, the anti-adhesive layer (2) is brought into direct contact with the wound to prevent the dressing from adhering to the wound, and the absorption layer (1) absorbs the blood or the tissue fluid. Furthermore, the absorption layer (1) may contain cotton, polyester fiber, polyurethane, alginic acid sodium salt, chitosan, or sodium carboxymethyl cellulose; the anti-adhesive layer (2) may be porous and contain polyurethane, polyethylene, polyvinyl chloride, or polyethylene terephthalate.

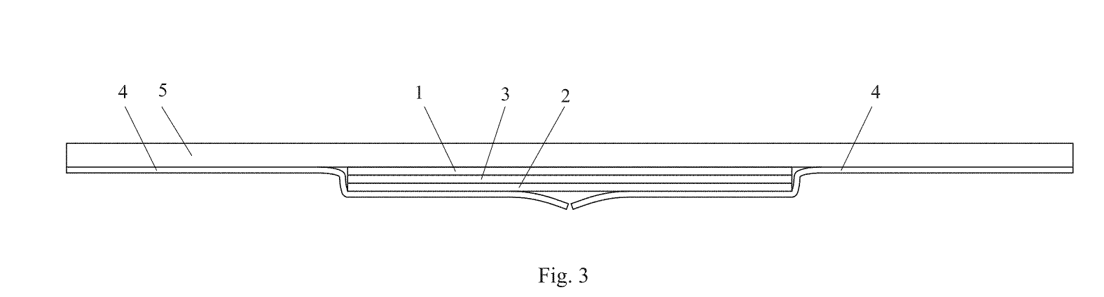

The laminating step (S7) can also be sequentially depositing an absorption layer (1), an antimicrobial layer (3) containing the antimicrobial composition, an anti-adhesive layer (2), and a release layer (4) on an adhesive layer (5), wherein the absorption layer (1) is in contact with a first portion of the adhesive layer (5), a first portion of the release layer (4) is in contact with the anti-adhesive layer (2), and a second portion of the release layer (4) is in contact with a second portion of the adhesive layer (5) to form an adhesive dressing (FIG. 3). Before the dressing is applied on a wound, the release layer (4) must be torn off. When the dressing is used, the anti-adhesive layer (2) is brought into direct contact with the wound to prevent the dressing from adhering to the wound, the second portion of the adhesive layer (5) is brought into direct contact with the skin to secure the dressing, and the absorption layer (1) absorbs the blood or the tissue fluid. Furthermore, the absorption layer (1) may contain cotton, polyester fiber, polyurethane, alginic acid sodium salt, chitosan, or sodium carboxymethyl cellulose; the anti-adhesive layer (2) may be porous and contain polyurethane, polyethylene, polyvinyl chloride, or polyethylene terephthalate; the release layer (4) may contain a waterproof silicon paper or a synthetic film of waterproof glue; the adhesive layer (5) may be waterproof or non-waterproof, and contain an adhesive-coated non-woven fabric, an adhesive-coated spunlace non-woven fabric, or an adhesive-coated hot rolled fabric.

The following examples are offered to further illustrate the foregoing embodiment:

### Example 1

First of all, 20 g silver nitrate was dissolved in 70 L of 25% ammonia aqueous solution. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 600° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,254 m²/g, a silver particle size of less than 50 nm, and a silver particle amount of 0.368 wt %.

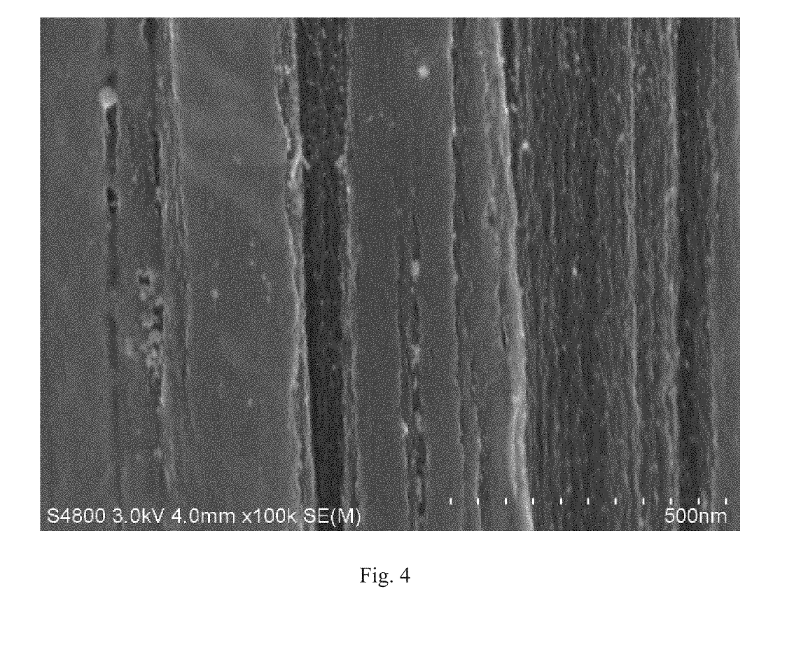

As shown in FIG. 4, the silver particle is uniformly distributed on the activated carbon fiber.

### Example 2

First of all, 20 g silver nitrate was dissolved in 70 L of 25% ammonia aqueous solution. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 800° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,198 m²/g, a silver particle size of less than 50 nm, and a silver particle amount of 0.325 wt %.

### Example 3

First of all, 20 g silver nitrate was dissolved in 70 L of 25% ammonia aqueous solution. A polyacrylonitrile (PAN)-based activated carbon fiber, which had a BET specific surface area of 1,082 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 600° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 920 m²/g, a silver particle size of less than 50 nm, and a silver particle amount of 0.346 wt %.

### Example 4

First of all, 20 g silver nitrate was dissolved in 70 L of 25% ammonia aqueous solution. A polyacrylonitrile (PAN)-based activated carbon fiber, which had a BET specific surface area of 1,082 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 800° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 875 m²/g, a silver particle size of less than 50 nm, and a silver particle amount of 0.302 wt %.

### Example 5

First of all, 20 g silver nitrate was dissolved in 70 L of 50% ammonia aqueous solution. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 600° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,196 m²/g, a silver particle size of less than 50 nm, and a silver particle amount of 0.385 wt %.

### Example 6

First of all, 20 g silver nitrate was dissolved in 70 L of 25% glacial acetic acid aqueous solution. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 600° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,283 m²/g, a silver particle size of less than 50 nm, and a silver particle amount of 0.432 wt %.

### Example 7

First of all, 20 g silver nitrate was dissolved in 70 L of aqueous solution containing 25% ammonia and 25% glacial acetic acid. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 800° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,250 m²/g, a silver particle size of less than 50 nm, and a silver particle amount of 0.392 wt %.

### Comparative Example 1

First of all, 20 g silver nitrate was dissolved in 70 L of water. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that a silver nitrate particle was attached to the activated carbon fiber. Hereafter, the combination of the activated carbon fiber and the silver nitrate particle was called “carbonaceous composition.” After cooling the silver nitrate particle to room temperature at a ramp down rate of 4° C./min, the carbonaceous composition was washed with water for 1 hour. Finally, the carbonaceous composition was thermally dried at 100° C. for 2 hours to remove the remaining liquid. At this moment, the carbonaceous composition had the following characteristics: a BET specific surface area of 1,480 m²/g, a silver nitrate particle size range in nanometer to lower micrometer, and a silver nitrate particle amount of 8.245 wt %.

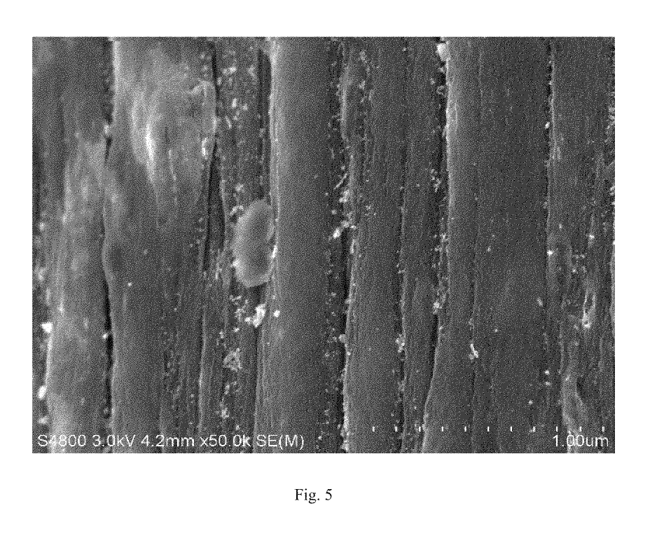

As shown in FIG. 5, the silver nitrate particle is not uniformly distributed on the activated carbon fiber.

### Comparative Example 2

First of all, 20 g silver nitrate was dissolved in 70 L of 25% ammonia aqueous solution. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that a silver nitrate particle was attached to the activated carbon fiber. Hereafter, the combination of the activated carbon fiber and the silver nitrate particle was called “carbonaceous composition.” After cooling the silver nitrate particle to room temperature at a ramp down rate of 4° C./min, the carbonaceous composition was washed with water for 1 hour. Finally, the carbonaceous composition was thermally dried at 100° C. for 2 hours to remove the remaining liquid. At this moment, the carbonaceous composition had the following characteristics: a BET specific surface area of 1,365 m²/g, a silver nitrate particle size range in nanometer to lower micrometer, and a silver nitrate particle amount of 3.184 wt %.

### Comparative Example 3

First of all, 20 g silver nitrate was dissolved in 70 L of 25% ammonia aqueous solution. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 300° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,308 m²/g, a silver particle size of less than 100 nm, and a silver particle amount of 0.632 wt %.

### Comparative Example 4

First of all, 20 g silver nitrate was dissolved in 70 L of 25% ammonia aqueous solution. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 450° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,286 m²/g, a silver particle size of less than 100 nm, and a silver particle amount of 0.522 wt %.

### Comparative Example 5

First of all, 20 g silver nitrate was dissolved in 70 L of water. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 600° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,346 m²/g, a silver particle size of less than 100 nm, and a silver particle amount of 0.531 wt %.

### Comparative Example 6

First of all, 20 g silver nitrate was dissolved in 70 L of water. An activated carbon fiber, which had a BET specific surface area of 1,538 m²/g and a weight of 400 g, was soaked in the thus-obtained silver nitrate aqueous solution under a stir for 2 hours. After which, the silver nitrate aqueous solution was thermally dried at 100° C. for 2 hours so that the silver nitrate was attached to the activated carbon fiber. After heating the silver nitrate to 450° C. at a ramp up rate of 2° C./min, the silver nitrate was stayed at this temperature for 1 hour so that the silver nitrate was converted into a silver particle attached to the activated carbon fiber. Hereafter, the combination of the activated carbon fiber and the silver particle was called “carbonaceous composition.” After cooling the silver particle to room temperature at a ramp down rate of 4° C./min, the carbonaceous composition was washed with water for 1 hour to remove any dissociated particle. Finally, the carbonaceous composition was thermally dried at 100° C. for 2 hours to remove the remaining liquid. At this moment, the carbonaceous composition had the following characteristics: a BET specific surface area of 1,142 m²/g, a silver particle size of less than 200 nm, and a silver particle amount of 0.645 wt %.

### Analysis 1

According to the American Association of Textile Chemists and Colorists (AATCC)-100 antimicrobial product test, various bacterial strains were incubated with an equivalent amount of the carbonaceous compositions obtained in all Examples and Comparative Examples for 24 hours so as to perform an antimicrobial analysis. As the results shown in Table 1, all the carbonaceous compositions have antimicrobial activity.

TABLE 1
Antimicrobial activity of each carbonaceous composition
after 24-hour incubation with bacteria

Sterilizing Rate(%)

| | Escherichia | Staphylococcus | Pseudomonas |
|---|---|---|---|
| | coli | aureus | aeruginosa |
| | (ATCC8739) | (ATCC33591) | (ATCC9027) |
| Example 1 | >99.99 | >99.99 | 99.98 |
| Example 2 | >99.99 | >99.99 | 99.98 |
| Example 3 | >99.99 | >99.99 | 99.98 |
| Example 4 | >99.99 | >99.99 | 99.98 |
| Example 5 | >99.99 | >99.99 | 99.98 |
| Example 6 | 99.98 | >99.99 | 99.98 |
| Example 7 | 99.98 | >99.99 | 99.98 |
| Comparative | 99.98 | 99.98 | 99.98 |
| Example 1 | | | |
| Comparative | 99.98 | 99.98 | 99.98 |
| Example 2 | | | |
| Comparative | 99.98 | 99.98 | 99.98 |
| Example 3 | | | |
| Comparative | 99.98 | 99.98 | 99.98 |
| Example 4 | | | |
| Comparative | 99.98 | 99.98 | 99.98 |
| Example 5 | | | |
| Comparative | 99.98 | 99.97 | 99.97 |
| Example 6 | | | |

### Analysis 2

According to the International Organization for Standardization (ISO) 10993-5: 2009 (E) standard, L929 mouse fibroblasts were incubated with an equivalent amount of the carbonaceous compositions obtained in all Examples and Comparative Examples for 24 hours so as to perform a toxicity analysis. As the results shown in Table 2, it is learned from the comparison of Examples 1-2 with Comparative Examples 1-6 that the use of the reductant and the proper range of the pyrolyzing temperature are in relation to the low cell toxicity of the carbonaceous compositions.

TABLE 2
Cell toxicity of each carbonaceous composition

| | The ratio of rounded or cracked cells(%) |
|---|---|
| Example 1 | 8 |
| Example 2 | 4 |
| Example 3 | 7 |
| Example 4 | 3 |
| Example 5 | 12 |
| Example 6 | 29 |
| Example 7 | 21 |
| Comparative | 72 |
| Example 1 | |
| Comparative | 58 |
| Example 2 | |
| Comparative | 32 |
| Example 3 | |
| Comparative | 28 |
| Example 4 | |
| Comparative | 22 |
| Example 5 | |
| Comparative | 33 |
| Example 6 | |

As described above, the manufacturing method of the foregoing embodiment can prevent the formation of a non-uniformly distributed agglomerate resulted from active ingredient salt aggregation on the carbonaceous material. This can make the active ingredient particle uniformly distributed on the carbonaceous material. Therefore, when the obtained antimicrobial composition is prepared as the dressing, the dressing can eliminate bacteria without harming a living body.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for manufacturing an antimicrobial composition, comprising:
providing an aqueous solution containing an active ingredient salt and a reductant;
immersing a carbonaceous material in the aqueous solution;
thermally drying the aqueous solution at 80-120° C. so as to attach the active ingredient salt to the carbonaceous material; and
pyrolyzing the active ingredient salt at 600-800° C. to convert into a particle of the active ingredient attached to the carbonaceous material so as to form the antimicrobial composition.

2. The method as claimed in claim 1, wherein the active ingredient salt is selected from the group consisting of a silver salt, a copper salt, a gold salt, a palladium salt, a zinc salt, a platinum salt, an aluminum salt, a nickel salt, a cobalt salt, a silicon salt, a calcium salt, a titanium salt, and a chromium salt.

3. The method as claimed in claim 1, wherein the reductant is selected from the group consisting of glacial acetic acid, ammonia water, ascorbic acid, and glucose.

4. The method as claimed in claim 1, wherein the carbonaceous material is selected from the group consisting of an activated carbon fiber, a carbon fiber, an activated carbon powder, a charcoal, a bamboo charcoal granule, a carbon black, a graphite powder, a carbon nanotube, a carbon nanopowder, a graphene, a swelling graphite powder, a carbon powder made from phenol formaldehyde resins, and a carbon powder made from artificial resins.

5. The method as claimed in claim 1, wherein the pyrolyzing temperature is of 600 or 800° C.

6. The method as claimed in claim 1, wherein in the immersing step, the salt is present in an amount of 0.01 wt %-1 wt %, the reductant is present in an amount of 1 wt %-50 wt %, and the carbonaceous material is present in an amount of 0.01 wt %-4 wt %, all based on total weight of the aqueous solution and the carbonaceous material.

7. A dressing, sequentially comprising:
an anti-adhesive layer;
an antimicrobial layer containing the antimicrobial composition manufactured by the method as claimed in claim 1; and
an absorption layer.

8. The dressing as claimed in claim 7, wherein the active ingredient salt is selected from the group consisting of a silver salt, a copper salt, a gold salt, a palladium salt, a zinc salt, a platinum salt, an aluminum salt, a nickel salt, a cobalt salt, a silicon salt, a calcium salt, a titanium salt, and a chromium salt.

9. The dressing as claimed in claim 7, wherein the reductant is selected from the group consisting of glacial acetic acid, ammonia water, ascorbic acid, and glucose.

10. The dressing as claimed in claim 7, wherein the anti-adhesive layer is porous and contains polyurethane, polyethylene, polyvinyl chloride, or polyethylene terephthalate.

11. The dressing as claimed in claim 7, wherein the absorption layer contains cotton, polyester fiber, polyurethane, alginic acid sodium salt, chitosan, or sodium carboxymethyl cellulose.

12. A dressing, sequentially comprising:
a release layer;
an anti-adhesive layer;
an antimicrobial layer containing the antimicrobial composition manufactured by the method as claimed in claim 1;
an absorption layer; and
an adhesive layer;
wherein the absorption layer is in contact with a first portion of the adhesive layer, a first portion of the release layer is in contact with the anti-adhesive layer, and a second portion of the release layer is in contact with a second portion of the adhesive layer.

13. The dressing as claimed in claim 12, wherein the active ingredient salt is selected from the group consisting of a silver salt, a copper salt, a gold salt, a palladium salt, a zinc salt, a platinum salt, an aluminum salt, a nickel salt, a cobalt salt, a silicon salt, a calcium salt, a titanium salt, and a chromium salt.

14. The dressing as claimed in claim 12, wherein the reductant is selected from the group consisting of glacial acetic acid, ammonia water, ascorbic acid, and glucose.

15. The dressing as claimed in claim 12, wherein the release layer contains a waterproof silicon paper or a synthetic film of waterproof glue.

16. The dressing as claimed in claim 12, wherein the anti-adhesive layer is porous and contains polyurethane, polyethylene, polyvinyl chloride, or polyethylene terephthalate.

17. The dressing as claimed in claim 12, wherein the absorption layer contains cotton, polyester fiber, polyurethane, alginic acid sodium salt, chitosan, or sodium carboxymethyl cellulose.

18. The dressing as claimed in claim 12, wherein the adhesive layer is waterproof or non-waterproof, and contains an adhesive-coated non-woven fabric, an adhesive-coated spunlace non-woven fabric, or an adhesive-coated hot rolled fabric.
